(19) United States
(12) Patent Application Publication
(10) Pub. No.: US 2019/0298685 A1
(43) Pub. Date: Oct. 3, 2019

(54) Process Of Improving Water Solubility Of Sesamin

(71) Applicant: Thailand Excellence Center For Tissue Engineering and Stem Cells, Chiang Mai (TH)

(72) Inventor: Prachya Kongtawelert, Chiang Mai (TH)

(21) Appl. No.: 16/344,686

(22) PCT Filed: Feb. 14, 2017
(86) PCT No.: PCT/TH2017/000003

§ 371 Date: Apr. 24, 2019

(51) Int. Cl. A61K 31/36; A61K 9/107; A61K 47/26; A61K 9/19; A61K 9/51

(52) U.S. Cl. CPC A61K 31/36; A61K 9/1075; A61K 9/5192; A61K 9/19; A61K 9/5161; A61K 47/26

(57) ABSTRACT

A process of improving water solubility of sesamin by creating a mixture of poloxamer and surfactant in appropriate proportion and sequence wherein the sesamin complex compounds derived from the process are at a nanometer (nm) scale and are highly soluble in water. Upon adding a maltodextrin into the mixture at a preferable speed and drying or freeze-drying said mixture thereafter, the resulting sesamin powders are highly stable and can actively release its medicinal effect for a long period of time.

### FIELD OF THE INVENTION

The invention relates to the fields of chemistry and pharmaceutical science with a special relation to a process of improving water solubility of sesamin by means of forming a complex composition with a poloxamer and a surfactant.

### BACKGROUND OF THE INVENTION

Sesamin, also chemically known as 5,5′-(1S,3aR,4S,6aR)-tetrahydro-1H,3H-furo[3,4-c]furan-1,4-diylbis(1,3-benzodioxole), is a chemical compound in a lignin group which can be found in sesame. There has been a report from a laboratory that sesamin can control fatty acid metabolism (Umeda-Sawada, R., Fujiwara, Y., Abe, H. & Seyama, Y. (2003) J. Nutr. Sci. Vitaminol. 49,442-446-10) and cholesterol (Kang Y P, Wang N H, Jou H J, Wang T A. (2006) J. Nutr. (136(5), 1270-1275) Further, it is known to prevent cancer (Harikumar K B, Sung B, Tharakan S T, Pandey M K, Joy B, Guha S, Krishnan S, Aggarwal B B, (2010) Mol. Cancer Research. 8(5): 751-761) and protect neurons from stress due to oxidation process (Hamada N., Fujita Y., Tanaka A., Naoi M., Nozawa Y., et al (2009) J Neural Transm 116: 841-852); further, it could aid the rehabilitation of cells inside the bone as well (Wanachewin O., Klangjorhor J., Pothacharoen P., Phitak T., Loahapoonrungsee A., Pruksakorn D., Kongtawelert P. (2015). J. Func. Food. 14: 395-406).Nevertheless, sesamin has limited water solubility and is soluble in ethanol, which is edible solvent at 0.5 per millilitres only.

To resolve the issue as stated above, a continuous liquid carbon dioxide system as be utilized to produce nano-particles of sesamin in one research report (Arita T., Manabe N., Nakahara K. (2012) Journal of Nanoparticle Research, 14(11), (2012): 1251); however, such system still incurred high costs and further required using specific equipment.

Presently, there has not been any system that can effectively improve water solubility of sesamin; namely, to improve sesamin solubility and control its release efficiently. Thus, the present invention shall utilize two pharmaceutical substances together in order to create a group of sesamin complex nano-particles that are water soluble and which can maintain water soluble property for a long period of time.

### SUMMARY OF THE INVENTION

The present invention is related to a creation of, sequentially, a complexation and micellization of sesamin with two supporting substances; namely, a complexing agent and a surfactant, to prevent rupturing of the sesamin complex compounds. The objective of the invention is to permanently improve the water solubility of sesamin wherein the resulting sesamin solution can slowly release sesamin at least 6-8 hours to solve any issues relating to dissolution of sesamin and capturing and releasing sesamin for improved medicinal or pharmaceutical effectiveness.

### BRIEF DESCRIPTION OF THE DRAWINGS

FIG. 1 illustrates one example of the sizes and size distribution of sesamin complex compounds according to this invention when a polysorbate 80 of 250 microliters (μL) per 200 micrograms (μg) of sesamin is used.

FIG. 2 illustrates one example of the zeta potential distribution of sesamin complex compounds according to this invention when a polysorbate 80 of 250 μL per 200 μg of sesamin is used.

FIG. 3 illustrates one example of the sizes and size distribution of sesamin complex compounds according to this invention when using the complex compounds of 5 mg with polysorbate 80 at 2500 μL per 200 μg of sesamin.

FIG. 4 illustrates one example of the zeta potential distribution of sesamin complex compounds according to this invention when using the complex compounds of 5 mg with polysorbate 80 at 250 μL per 200 μg of sesamin.

FIG. 5 illustrates one example of the sizes and size distribution of sesamin complex compounds according to this invention when using the complex compounds of 7.5 mg with polysorbate 80 at 250 μL per 200 μg of sesamin.

FIG. 6 illustrates one example of the zeta potential distribution of sesamin complex compounds according to this invention when using the complex compounds of 7.5 mg with polysorbate 80 at 250 μL per 200 μg of sesamin.

FIG. 7 illustrates one example of the sizes and size distribution of sesamin complex compounds according to this invention when using the complex compounds of 10 mg with polysorbate 80 at 250 μL per 200 μg of sesamin.

FIG. 8 illustrates one example of the zeta potential distribution of sesamin complex compounds according to this invention when using the complex compounds of 10 mg with polysorbate 80 at 250 μL per 200 μg of sesamin.

FIG. 9 illustrates one example of the sizes and size distribution of sesamin complex compounds according to this invention when using the complex compounds of 12.5 mg with polysorbate 80 at 250 μL per 200 μg of sesamin.

FIG. 10 illustrates one example of the zeta potential distribution of sesamin complex compounds when using the complex compounds of 12.5 mg with polysorbate 80 at 250 μL per 200 μg of sesamin.

FIG. 11 illustrates one example of the sizes and size distribution of sesamin complex compounds according to this invention when using the complex compounds of 15 mg with polysorbate 80 at 250 μL per 200 μg of sesamin.

FIG. 12 illustrates one example of the zeta potential distribution of sesamin complex compounds according to this invention when using the complex compounds of 15 mg with polysorbate 80 at 250 μL per 200 μg of sesamin.

FIG. 13 illustrates one example of the sizes and size distribution of sesamin complex compounds according to this invention when using the complex compounds of 17.5 mg with polysorbate 80 at 250 μL per 200 μg of sesamin. FIG. 14 illustrates one example of the zeta potential distribution of sesamin complex compounds according to this invention when using the complex compounds of 17.5 mg with polysorbate 80 at 250 μL per 200 μg of sesamin.

FIG. 15 illustrates one example of the sizes and size distribution of sesamin complex compounds according to this invention when using the complex compounds of 20 mg with polysorbate 80 at 250 μL per 200 μg of sesamin.

FIG. 16 illustrates one example of the zeta potential distribution of sesamin complex compounds according to this invention when using the complex compounds of 20 mg with polysorbate 80 at 250 μL per 200 μg of sesamin.

FIG. 17 illustrates one example of a table showing the effects of various amounts of poloxamers on sizes and size distribution of sesamin nano-particles.

FIG. 18 illustrates one example of rates of releases of sesamin nano-particles in a 10 mM HEPES solution (pH 7.4).

### DETAILED DESCRIPTION OF THE INVENTION

A process of improving water solubility of sesamin according to this invention generally comprising the steps of inducing a creation of complex compounds of sesamin with a poloxamer-based complexing agent and wrapping a surfactant around said complex compounds and creating micelles wherein the dissolution is self-generated under a suitable condition inducing by two solutions, namely the sesamin and poloxamer, to generate a stable mixture solution.

A process of preparing a set of sesamin complex compounds specifically comprising the use of a complex solution of the poloxamer group-to-sesamin at the ratio of 0.1-2:1-10 by mass, preferably 0.5-2:1-10 by mass, and a surfactant at the amount of no less than 200 microliters (μL), preferably 230-270 μL, per 1 milligram (mg) of sesamin.

A process of dissolving sesamin according to this invention comprising the steps of dissolving sesamin in an organic solvent, preferably chosen from chloroform, ethanol, methanol, and DMSO, at a sesamin-to-solvent ratio of 1-5:500-1,500 (mass:volume), preferably of 3:1,000 (mass:volume), and at the same time or sequentially, preparing a poloxamer solution in water wherein the amount of poloxamer used is at least 1-10 times of the weight of sesamin in the above solution. Subsequently, a poloxamer solution is added or dropped, wherein the poloxamer is preferably chosen from a poloxamer 127, a poloxamer 80, or a derivative of any of the two, but most preferably poloxamer F127, into a sesamin solution until a clear or transparent mixture is produced, either by hand or equipment for mixing the solutions together at a preferable speed The surfactant, preferably chosen from an ionic or a nonionic surfactant, but most preferably a nonionic surfactant consisting of a water soluble polysorbate group or a polysorbate 80, is then added or dropped into the mixture at the amount of no less than 100-300 μL per 200 μg of sesamin, but preferably at 230-270 μL per 1 mg of sesamin and/or 1-15 mg of poloxamer, which shall depend on the molecular weight of the poloxamer solution used. The mixture is then stirred by either hand or equipment for stirring at high-speed in order to distribute the surfactant throughout the mixture and to produce a white or cloudy mixture solution. Additionally, a maltodextrin of 1-15% by weight is added into the mixture and are then mixed at high-speed to dissolve said maltodextrin in the mixture. The derived mixture solution is then centrifuged at the speed of 10,000-15,000 rounds per minute, preferably at 12,500 rounds per minute and is freeze dried or lyophilized in order to eliminate any water and organic solvent and to attain a final sesamin product with high stability and water solubility

Such final sesamin product according to this invention, when mixed with water, is found to be highly soluble; namely, a sesamin at 200 μg can completely dissolve in water by using no more than 1 mL and which is characterized by a, homogenous, white solution without separating into layers or without precipitation.

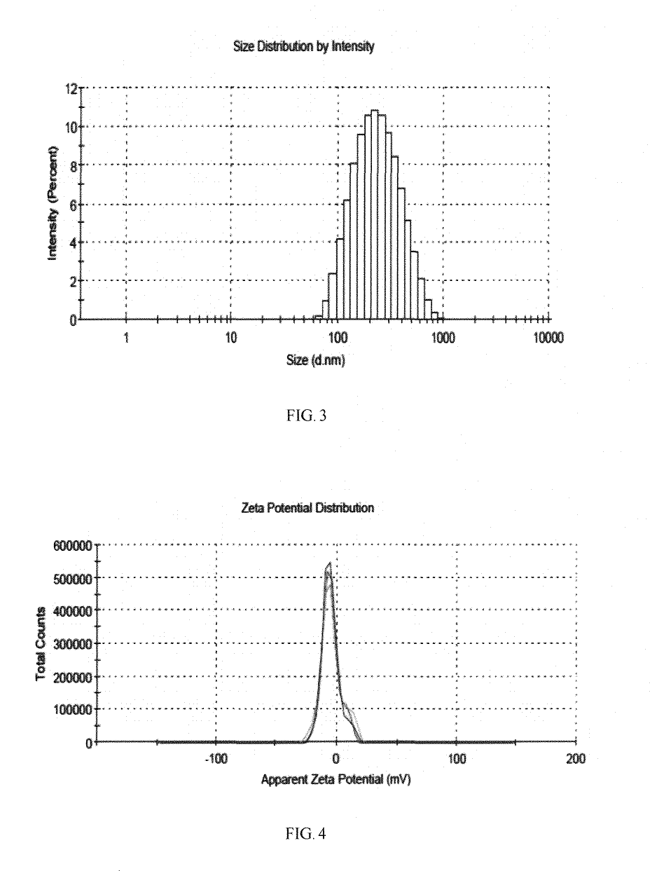

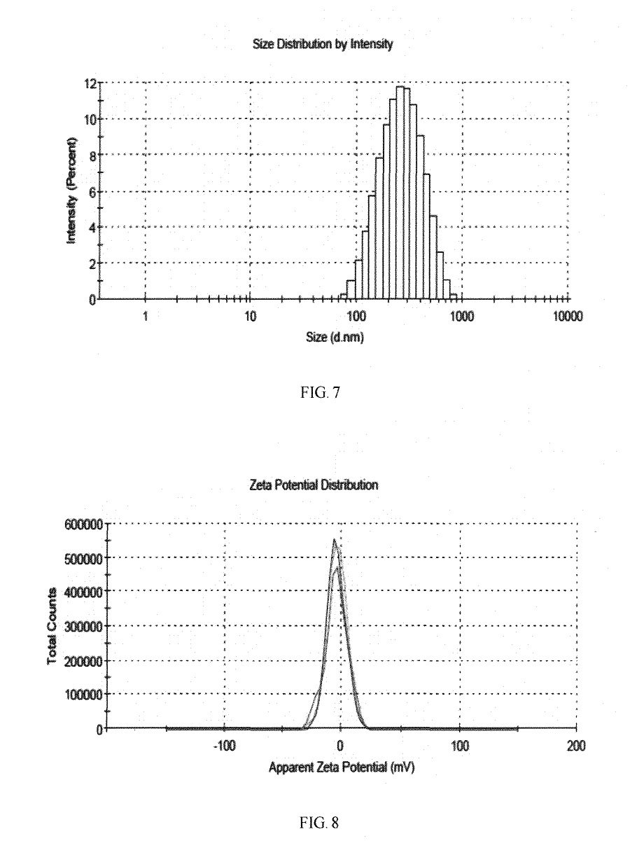

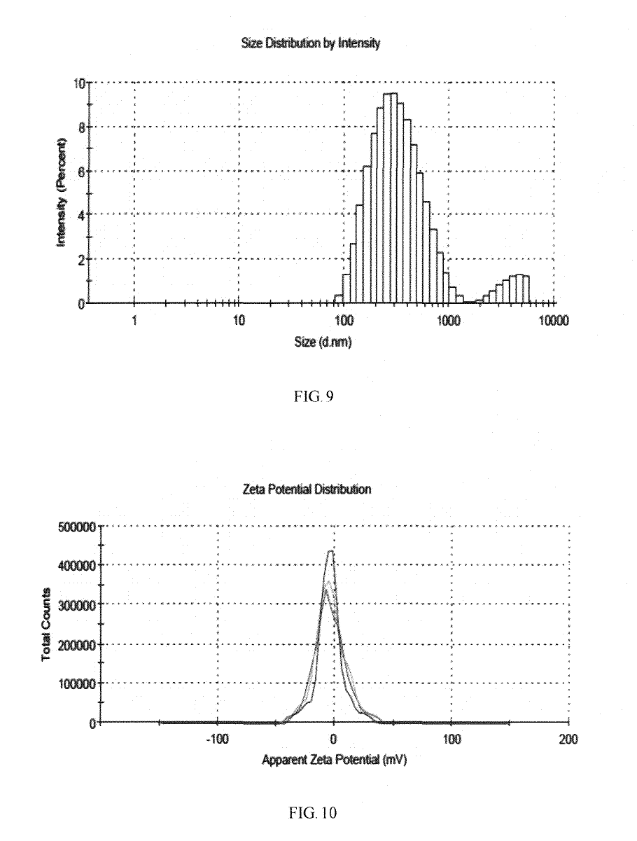

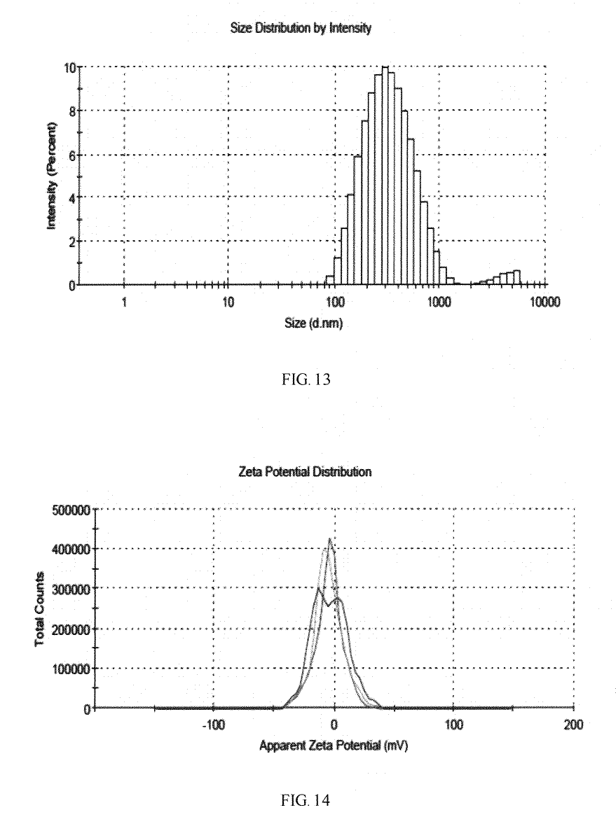

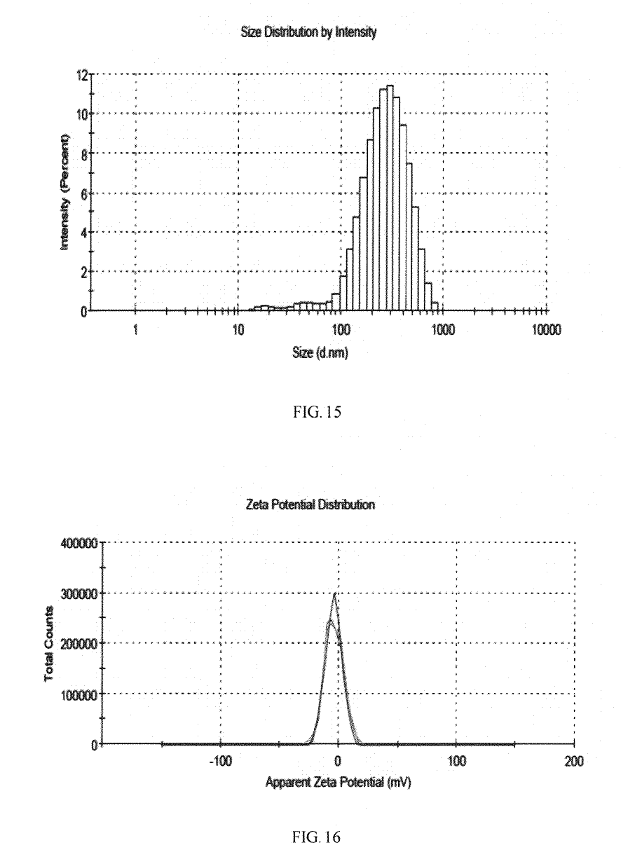

The increased solubility above is induced by the initial complex compositions of sesamin molecules and poloxamer, and then by having the surfactant molecules wrapping around the initial complex compositions to create micelles in various sizes depending on the types of poloxamer and surfactant used to create micelles at nanometer scale and which can be measured by equipment, such as a photon correlation spectrometer, as illustrated in FIGS. 1, 3, 5, 7, 9, 11, 13, and 15. FIG. 17 are examples of the values derived from the effects of various amounts of poloxamers used on sizes and size distribution of sesamin nanoparticles.

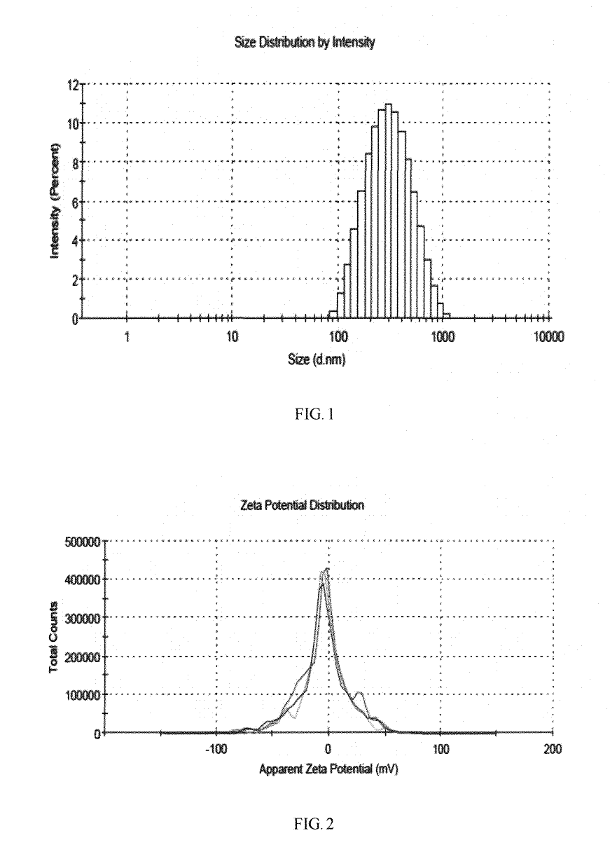

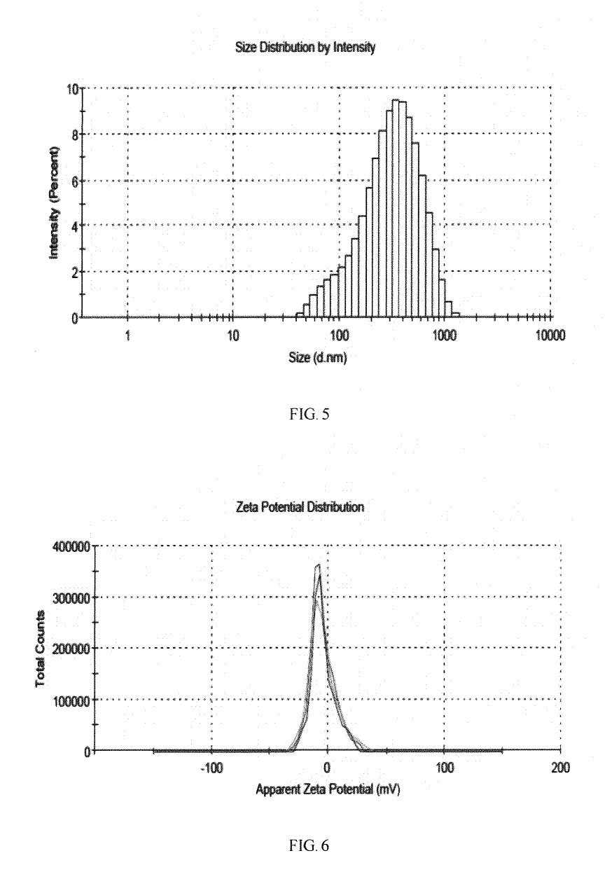

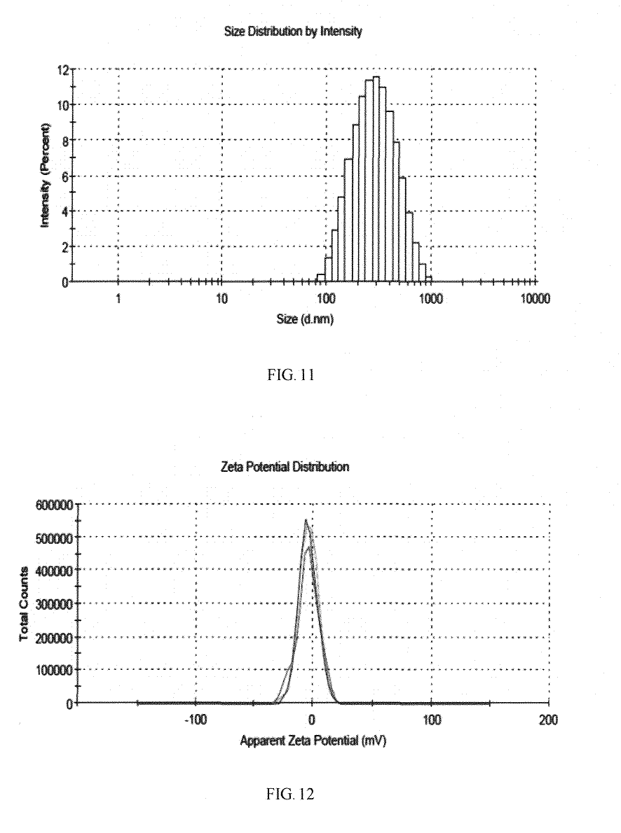

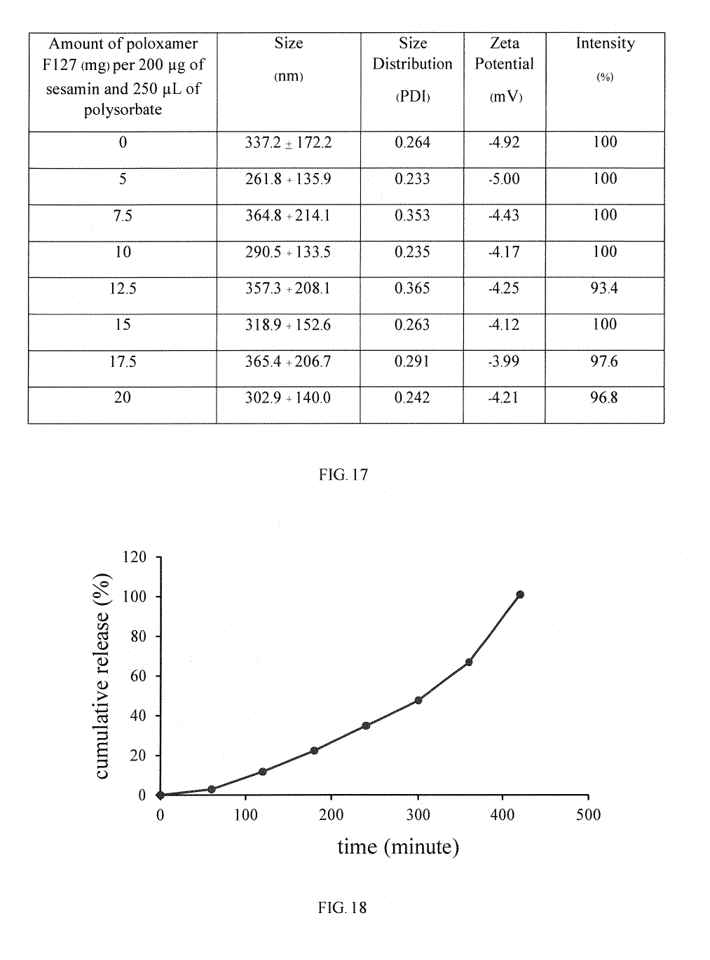

When the derived solution comprising a set of polysorbate 80 at 100, 200, and 300 μL are completed separated from water and organic solvent by means of evaporation under pressure and freeze drying, the resulting product is highly soluble. When tested with 1 μL to 5 mL, the complex sesamin compounds can completely dissolve in the polysorbate 80 mixture from 100 μL and above wherein the sizes of the particles and zeta potential of the particle surfaces can be found in FIGS. 1-16 with the summary of the results in FIG. 17. It is found, in one example, that utilizing polysorbate 80 from 100 μL can maximize the dissolution of sesamin particles with maximum stability and can retain more than 70% of sesamin. In summary, it is found that sesamin according to this invention can completely dissolve in water and can be kept or stored in nanoparticle forms can be slowly release sesamin up to 7-8 hours as can be depicted in FIG. 18.

Although this invention has been disclosed in the context of certain embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. Thus, it is intended that the scope of the present invention herein disclosed should not be limited by the particular disclosed embodiments described above, but should be determined only by a fair reading of the claims that follow.

1. A process of improving water solubility of sesamin by a creation of complex compounds and micellization of sesamin with a complexing agent and a surfactant wherein the sesamin complex compounds comprising a poloxamer based complexing agent at a ratio of 0.5-2:1-10 by mass and a surfactant of no less than 200 μL per 1 mg of sesamin.

2. The process of improving water solubility of sesamin according to claim 1 wherein a maltodextrin of 1-15% by weight is further added into the complex compounds for drying or freeze-drying or lyophilizing purpose.

3. The process of improving water solubility of sesamin according to claim 1 wherein the poloxamer used is chosen from a poloxamer 127, a poloxamer 68, or a derivative of either the poloxamer 127 or poloxamer 68 thereof.

4. The process of improving water solubility of sesamin according to claim 3 wherein the poloxamer used is preferably a poloxamer F127.

5. The process of improving water solubility of sesamin according to claim 1 wherein the surfactant is chosen from an ionic surfactant or a nonionic surfactant.

6. The process of improving water solubility of sesamin according to claim 5 wherein the surfactant is preferably nonionic surfactant consisting of a water soluble polysorbate group of surfactant.

7. The process of improving water solubility of sesamin according to claim 6 wherein the polysorbate surfactant is preferably polysorbate 80.

8. The process of improving water solubility of sesamin according to claim 1 wherein the surfactant used is preferably 230-270 μL per 1 mg of sesamin or 1-15 mg of poloxamer.

9. The process of improving water solubility of sesamin according to claim 1 wherein the process of preparing the sesamin complex compounds comprises:
(a): dissolving sesamin in an organic solvent at a sesamin-to-solvent ratio of 1-5:500-1,500 (mass:volume);
(b): preparing the poloxamer in water wherein the amount of poloxamer is 1-10 times of the mass of sesamin solution from (a);
(c): dropping the poloxamer solution into the sesamin solution and mixing them together either by hand or by a mixing equipment until the mixture turns clear;
(d): dropping the surfactant into the mixture from (c) and mixing them together either by hand or mixing equipment to disintegrate or distribute the surfactant into the mixture until the mixture turns white or cloudy;
(e): adding 1-15% of maltodextrin into the mixture and mixing them together to dissolve said maltodextrin; and
(f): drying or freeze-drying or lyophilizing said mixture from (e) to discard water and organic solvent from the final sesamin product.

10. The process of improving water solubility of sesamin according to claim 9 wherein the organic solvent of (a) is chosen from a chloroform, a methanol, an ethanol, an olive oil, or a DMSO.

11. The process of improving water solubility of sesamin according to claim 9 wherein the mixture of (f) is centrifuged at the speed of 10,000-15,000 rounds per minute and is dried, freeze dried, or lyophilized in order to eliminate any water and organic solvent from the final sesamin product.
